(19) United States
(12) Patent Application Publication
(10) Pub. No.: US 2013/0273137 A1
(43) Pub. Date: Oct. 17, 2013

(54) DRUG DELIVERY COATING AND DEVICES

(71) Applicants: Kenneth J. Mandell, Arlington, MA (US); Paula T. Hammond, Newton, MA (US); Renee C. Fuller, Las Vegas, NV (US); Joseph F. Rizzo, III, Newton, MA (US); Anita Shukla, Cambridge, MA (US)

(72) Inventors: Kenneth J. Mandell, Arlington, MA (US); Paula T. Hammond, Newton, MA (US); Renee C. Fuller, Las Vegas, NV (US); Joseph F. Rizzo, III, Newton, MA (US); Anita Shukla, Cambridge, MA (US)

(73) Assignees: MASSACHUSETTS EYE & EAR INFIRMARY, Boston, MA (US); Massachusetts Institute of Technology, Cambridge, MA (US)

(21) Appl. No.: 13/695,836

(22) PCT Filed: May 3, 2011
(86) PCT No.: PCT/US11/35057

§ 371 Date: Jun. 7, 2013

Related U.S. Application Data
(60) Provisional application No. 61/330,865, filed on May 3, 2010.

(51) Int. Cl. A61L 27/28; A61F 2/16; A61L 27/54

(52) U.S. Cl. CPC A61L 27/28; A61L 27/54; A61F 2/16
USPC 424/428; 424/400; 514/20.9; 514/567

(57) ABSTRACT

In various embodiments, the present invention provides certain systems comprising a multi-layer decomposable film coating composition on a substrate, where the coating composition includes one or more releasable agents in at least one of its layers, and decomposes layer-by-layer to release such agent(s) over time. In some embodiments, an intra-ocular lens (IOL) system comprising an IOL coated with a multi-layer decomposable film coating composition is disclosed.

### RELATED REFERENCES

This application claims priority to U.S. provisional patent application Ser. No. 61/330,865, filed May 3, 2010, the entire contents of which are herein incorporated by reference.

### GOVERNMENT FUNDING

The work described herein was supported, in part, by a grant from the National Institutes of Health (5-R01-AG029601). The Government of the United States has certain rights in this application.

### BACKGROUND

It is often desirable to delivery drugs from medical devices that are used in association with a body, and particularly medical devices that are implanted in a body. For example, such devices, can create infection, inflammation or other risks for subjects. Additionally, such devices are by their nature localized in or on a body, and can act as useful systems for local administration of therapeutic or other agents.

For example, a commonly performed intra-ocular surgery is cataract extraction in which an opacified lens is removed. The natural lens is routinely replaced with an artificial implantable intra-ocular lens (IOL) as is well known in the art. To combat the inflammation and potential infection, anti-inflammatory and antibiotic eye drops are routinely used after cataract extraction, usually for a period of a month or longer. Moreover, it is difficult to deliver effective doses of drugs to the posterior part of the eye. Drugs can be applied either topically or systemically or by local injection. In the case of systemic administration large doses of the drug are needed to penetrate thought the blood-retinal barrier, resulting often in side effects. Topical instillation of drugs has little therapeutic effect due to the poor penetration onto the posterior part of the eye. Intravitreous injection requires frequent injections in the vitreous to maintain the concentration of a drug within a therapeutic range over a long period of time and sometimes cause complications, such as vitreous haemorrhage, retinal detachment, or endophthalmitis.

To overcome the drawbacks of these conventional treatments, a controlled drug release system (e.g., coated IOL devices) may provide solution to control postoperative inflammation following surgery (e.g., a cataract surgery).

### SUMMARY

The present invention provides certain systems comprising a multi-layer decomposable film coating composition on a substrate, where the coating composition includes one or more therapeutic or other agents in at least one of its layers, and decomposes layer-by-layer to release such agent(s) over time.

In one aspect, the invention provides intra-ocular lens (IOL) systems comprising an IOL coated with a multi-layer decomposable film coating composition.

In one aspect, the invention provides systems comprising a multi-layer decomposable film coating composition on a substrate, wherein the multi-layer decomposable film coating composition itself comprises a plurality of multi-layer decomposable structures, each of which includes a different releasable agent or agents.

In some embodiments, the substrate included in provided systems is or comprises a device arranged and constructed for contact with a body (i.e., “bodily devices”). In some embodiments, the substrate included in provided systems is or comprises an implantable device. In some embodiments, the substrate included in provided systems is or comprises an IOL. Among other things, the present invention demonstrates and achieves various improvements in bodily devices, and particularly in delivery of agents from bodily devices. The present invention also encompasses the recognition that, in many cases, improvements to bodily devices can be achieved through use of a multi-layer decomposable film coating composition as described herein without requiring significant changes to structure and/or materials utilized in the bodily device. This feature renders the teachings of the present invention readily adaptable to a variety of contexts and substrates with modest and/or routine effort.

In some embodiments, provided systems comprise one or more anti-infective agents and/or one or more anti-inflammatory agents.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### DEFINITIONS

In order for the present invention to be more readily understood, certain terms are first defined below. Additional definitions for the following terms and other terms are set forth throughout the specification.

“Associated”: As used herein, the terms “associated”, “conjugated”, “linked”, “attached”, “complexed”, and “incorporated,” and grammatic equivalents, typically refer to two or more moieties connected with one another, either directly or indirectly (e.g., via one or more additional moieties that serve as a linking agent), to form a structure that is sufficiently stable so that the moieties remain connected under the conditions in which the structure is used, e.g., physiological conditions. In some embodiments, the moieties are associated to one another by one or more covalent bonds. In some embodiments, the moieties are associated to one another by a mechanism that involves specific (but non-covalent) binding (e.g. streptavidin/avidin interactions, antibody/antigen interactions, etc.). Alternatively or additionally, a sufficient number of weaker interactions (non-covalent) can provide sufficient stability for moieties to remain connected. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Biomolecules”: The term “biomolecules”, as used herein, refers to molecules (e.g., proteins, amino acids, peptides, polynucleotides, nucleotides, carbohydrates, sugars, lipids, nucleoproteins, glycoproteins, lipoproteins, steroids, etc.) whether naturally-occurring or artificially created (e.g., by synthetic or recombinant methods) that are commonly found in cells and tissues. Specific classes of biomolecules include, but are not limited to, enzymes, receptors, neurotransmitters, hormones, cytokines, cell response modifiers such as growth factors and chemotactic factors, antibodies, vaccines, haptens, toxins, interferons, ribozymes, anti-sense agents, plasmids, DNA, and RNA,

“Biocompatible”: The term “biocompatible”, as used herein is intended to describe materials that do not elicit a substantial detrimental response in vivo. In some embodiments, a substance is considered to be “biocompatible” if its addition to cells in vitro or in vivo results in less than or equal to about 50%, about 45%, about 40%, about 35%, about 30%, about 25%, about 20%, about 15%, about 10%, about 5%, or less than about 5% cell death.

“Biodegradable”: As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

“Hydrolytically degradable”: As used herein, “hydrolytically degradable” polymers are polymers that degrade fully in the sole presence of water. In preferred embodiments, the polymers and hydrolytic degradation byproducts are biocompatible. As used herein, the term “non-hydrolytically degradable” refers to polymers that do not fully degrade in the sole presence of water.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 7.0 to 7.4.

“Polyelectrolyte” or “polyion”: The terms “polyelectrolyte” or “polyion”, as used herein, refer to a polymer which under some set of conditions (e.g., physiological conditions) has a net positive or negative charge. Polycations have a net positive charge and polyanions have a net negative charge. The net charge of a given polyelectrolyte or polyion may depend on the surrounding chemical conditions, e.g., on the pH.

“Polynucleotide”, “nucleic acid”, or “oligonucleotide”: The terms “polynucleotide”, “nucleic acid”, or “oligonucleotide” refer to a polymer of nucleotides. The terms “polynucleotide”, “nucleic acid”, and “oligonucleotide”, may be used interchangeably. Typically, a polynucleotide comprises at least three nucleotides. DNAs and RNAs are polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

“Polypeptide”, “peptide”, or “protein”: According to the present application, a “polypeptide”, “peptide”, or “protein” comprises a string of at least three amino acids linked together by peptide bonds. The terms “polypeptide”, “peptide”, and “protein”, may be used interchangeably. Peptide may refer to an individual peptide or a collection of peptides. Inventive peptides preferably contain only natural amino acids, although non-natural amino acids (i.e., compounds that do not occur in nature but that can be incorporated into a polypeptide chain; see, for example, http://www.cco.caltech.edu/˜dadgrp/Unnatstruct.gif, which displays structures of non-natural amino acids that have been successfully incorporated into functional ion channels) and/or amino acid analogs as are known in the art may alternatively be employed. Also, one or more of the amino acids in an inventive peptide may be modified, for example, by the addition of a chemical entity such as a carbohydrate group, a phosphate group, a farnesyl group, an isofarnesyl group, a fatty acid group, a linker for conjugation, functionalization, or other modification, etc. In a preferred embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide. The phrase “enzyme polypeptide” refers to a polypeptide having enzymatic activity.

“Polysaccharide”, “carbohydrate” or “oligosaccharide”: The terms “polysaccharide”, “carbohydrate”, or “oligosaccharide” refer to a polymer of sugars. The terms “polysaccharide”, “carbohydrate”, and “oligosaccharide”, may be used interchangeably. Typically, a polysaccharide comprises at least three sugars. The polymer may include natural sugars (e.g., glucose, fructose, galactose, mannose, arabinose, ribose, and xylose) and/or modified sugars (e.g., 2′-fluororibose, 2′-deoxyribose, and hexose).

“Small molecule”: As used herein, the term “small molecule” is used to refer to molecules, whether naturally-occurring or artificially created (e.g., via chemical synthesis), that have a relatively low molecular weight. Typically, small molecules are monomeric and have a molecular weight of less than about 1500 g/mol. Preferred small molecules are biologically active in that they produce a local or systemic effect in animals, preferably mammals, more preferably humans. In certain preferred embodiments, the small molecule is a drug. Preferably, though not necessarily, the drug is one that has already been deemed safe and effective for use by the appropriate governmental agency or body. For example, drugs for human use listed by the FDA under 21 C.F.R. §§330.5, 331 through 361, and 440 through 460; drugs for veterinary use listed by the FDA under 21 C.F.R. §§500 through 589, incorporated herein by reference, are all considered acceptable for use in accordance with the present application.

“Substantial” or “substantive”: As used herein, the terms “substantial” or “substantive” and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Therapeutic agent”, “medication” or “drug”: As used herein, the phrases “therapeutic agent”, “medication”, or “drug” may be used interchangeably. They refer to any agent that, when administered to a subject, has a therapeutic effect and/or elicits a desired biological and/or pharmacological effect.

“Treating:” As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### BRIEF DESCRIPTION OF THE FIGURES

FIG. 1A and FIG. 1B are a schematic cross-sectional view of an eye and a schematic illustration of an IOL with non-limiting features, respectively.

FIG. 2 depicts a chemical structure of an exemplary polymer that may be used in accordance with the present invention. Shown is the structure for a poly 2 as used in Examples.

FIG. 3 illustrates exemplary LbL film architectures. A.) Antibiotic-only and NSAID-only LbL film architectures. B.) Composite antibiotic and NSAID LbL film architectures.

FIG. 4 illustrates exemplary results of solution based film component interactions. A.) Vancomycin-polyCD interaction. B.) Vancomycin-diclofenac interaction. All interactions were studied at four conditions: 0.1 M sodium acetate buffer and 1 M NaCl, pH 5 and 6.

FIG. 5 illustrate a typical study of diffusion and exchange behavior in single-therapeutic films.

FIG. 6 illustrates exemplary composite film drug release profiles. A.) Drug release from dipped LbL film: (poly 2/dextran sulfate/vancomycin/dextran sulfate)₆₀+(poly 2/polyCD-diclofenac)₂₀. B.) Drug release from sprayed LbL film: (poly 2/chondroitin sulfate/vancomycin/chondroitin sulfate)₆₀+(poly 2/polyCD-diclofenac)₂₀.

FIG. 7 illustrate exemplary total drug release from composite film architectures. A.) (Poly 2/chondroitin sulfate/vancomycin/chondroitin sulfate)₆₀+(poly 2/polyCD-diclofenac)₂₀ dipped. B.) (Poly 2/polyCD-diclofenac)₂₀+(poly 2/chondroitin sulfate/vancomycin/chondroitin sulfate)₆₀ dipped. C.) (Poly 2/alginate/vancomycin/alginate)₆₀+(poly 2/polyCD-diclofenac)₂₀ dipped. D.) (Poly 2/polyCD-diclofenac)₂₀+(poly 2/alginate/vancomycin/alginate)₆₀ dipped. E.) (Poly 2/polyCD-diclofenac)₂₀+(poly 2/dextran sulfate/vancomycin/dextran sulfate)₆₀ dipped. F.) (Poly 2/polyCD-diclofenac)₂₀+(poly 2/chondroitin sulfate/vancomycin/chondroitin sulfate)₆₀ sprayed

FIG. 8 shows typical multi-drug release device coatings. Scanning electron microscopy images of coated medical devices (scale bar=20 μm for IOL and bandage; 100 μm for suture). The uncoated IOL image shows both the lens (i.e., optic) and haptic regions. The visible crack on the coated IOL is a scratch on the film showing the existence of a smooth film on the lens.

FIG. 9 illustrates exemplary results of composite film-released drug efficacy. A.) COX activity of diclofenac released from LbL bandage coating at Day 1, 2, 4, and 6 of release. Controls of pure polyCD, pure vancomycin, and pure diclofenac were also included. B.) Vancomycin activity against agar coated S. aureus of (i) LbL coated bandage, (ii) uncoated bandage, and (iii) vancomycin control (30 μg) (scale bar=9 mm). C.) Normalized S. aureus inhibition by vancomycin released from dipped LbL film architecture: (poly 2/dextran sulfate/vancomycin/dextran sulfate)₆₀+(poly 2/polyCD-diclofenac)₂₀.

### DETAILED DESCRIPTION OF CERTAIN EMBODIMENTS

In various embodiments, compositions and methods for associating one or more releasable agents into a multi-layer decomposable film are disclosed. Provided composition and methods can be used to coat a substrate (e.g., bodily devices such as IOLs) for controlled release of one or more agents.

### Decomposable Films

Decomposition of the films is characterized by the substantially sequential degradation of at least a portion of the polyelectrolyte layers that make up the thin films. The degradation may be at least partially hydrolytic, at least partially enzymatic, at least partially thermal, and/or at least partially photolytic.

Decomposable films may have various thickness depending on methods of fabricating and applications. In some embodiments, a decomposable film has an average thickness in a range of about 1 nm and about 100 μm. In some embodiments, a decomposable film has an average thickness in a range of about 1 μm and about 50 μm. In some embodiments, a decomposable film has an average thickness in a range of about 2 μm and about 5 μm. In some embodiments, the average thickness of a decomposable film is or more than about 1 nm, about 100 nm, about 500 nm, about 1 μm, about 2 μm, about 3 μm, about 4 μm, about 5 μm, about 10 μm, bout 20 μm, about 50 μm, about 100 μm. In some embodiments, a decomposable film has an average thickness in a range of any two values above.

Decomposable films may be comprised of multilayer units with alternating layers of opposite charge, such as alternating anionic and cationic layers. At least one of the layers in a decomposable film includes a degradable polyelectrolyte. In some embodiments, a decomposable film include a plurality of polyelectrolyte layers. In some embodiments, a decomposable film include a plurality of a single unit (e.g., a bilayer unit, a tetralayer unit, etc.). In some embodiments, a decomposable film is a composite that include more than one units. For example, more than one units can have be different in film materials (e.g., polymers), film architecture (e.g., bilayers, tetralayer, etc.), film thickness, and/or releasable agents that are associate with one of the units. In some embodiments, a decomposable film is a composite that include more than one bilayer units, more than one tetralayer units, or any combination thereof. In some embodiments, a decomposable film is a composite that include a plurality of a single bilayer unit and a plurality of a single tetralayer unit (e.g. exemplary composite films as shown in Example 3 below).

Decomposable films for drug release in accordance with the present invention comprise releasable agents. In some embodiments, a decomposable film include more than one bilayer units and more than one releasable agents. In some embodiments, a decomposable film include more than one tetralayer units and more than one releasable agents. In some embodiments, a decomposable film include at least one bilayer unit, at least tetralayer unit, and more than one releasable agents.

Decomposable films may be exposed to a liquid medium (e.g., intracellular fluid, interstitial fluid, blood, intravitreal fluid, intraocular fluid, gastric fluids, etc.). In some embodiments, a decomposable film comprises at least one polycationic layer that degrades and at least one polyanionic layer that delaminates sequentially. Releasable agents are thus gradually and controllably released from the decomposable film. It will be appreciated that the roles of the layers of a decomposable film can be reversed. In some embodiments, a decomposable film comprises at least one polyanionic layer that degrades and at least one polycationic layer that delaminates sequentially. Alternatively, polycationic and polyanionic layers may both include degradable polyelectrolytes.

Degradable polyelectrolytes and their degradation byproducts may be biocompatible so as to make decomposable films amenable to use in vivo.

Degradable Polyelectrolytes

Any degradable polyelectrolyte can be used in the thin film disclosed herein, including, but not limited to, hydrolytically degradable, biodegradable, thermally degradable, and photolytically degradable polyelectrolytes. Hydrolytically degradable polymers known in the art include for example, certain polyesters, polyanhydrides, polyorthoesters, polyphosphazenes, and polyphosphoesters. Biodegradable polymers known in the art, include, for example, certain polyhydroxyacids, polypropylfumerates, polycaprolactones, polyamides, poly(amino acids), polyacetals, polyethers, biodegradable polycyanoacrylates, biodegradable polyurethanes and polysaccharides. For example, specific biodegradable polymers that may be used include but are not limited to polylysine, poly(lactic acid) (PLA), poly(glycolic acid) (PGA), poly(caprolactone) (PCL), poly(lactide-co-glycolide) (PLG), poly(lactide-co-caprolactone) (PLC), and poly(glycolide-co-caprolactone) (PGC). Those skilled in the art will recognize that this is an exemplary, not comprehensive, list of biodegradable polymers. The properties of these and other polymers and methods for preparing them are further described in the art. See, for example, U.S. Pat. Nos. 6,123,727; 5,804,178; 5,770,417; 5,736,372; 5,716,404 to Vacanti; 6,095,148; 5,837,752 to Shastri; 5,902,599 to Anseth; 5,696,175; 5,514,378; 5,512,600 to Mikos; 5,399,665 to Barrera; 5,019,379 to Domb; 5,010,167 to Ron; 4,806,621; 4,638,045 to Kohn; and 4,946,929 to d'Amore; see also Wang et al., J. Am. Chem. Soc. 123:9480, 2001; Lim et al., J. Am. Chem. Soc. 123:2460, 2001; Langer, Acc. Chem. Res. 33:94, 2000; Langer, J. Control Release 62:7, 1999; and Uhrich et al., Chem. Rev. 99:3181, 1999. Of course, co-polymers, mixtures, and adducts of these polymers may also be employed.

Anionic polyelectrolytes may be degradable polymers with anionic groups distributed along the polymer backbone. Anionic groups, which may include carboxylate, sulfonate, sulphate, phosphate, nitrate, or other negatively charged or ionizable groupings, may be disposed upon groups pendant from the backbone or may be incorporated in the backbone itself. Cationic polyelectrolytes may be degradable polymers with cationic groups distributed along the polymer backbone. Cationic groups, which may include protonated amine, quaternary ammonium or phosphonium-derived functions or other positively charged or ionizable groups, may be disposed in side groups pendant from the backbone, may be attached to the backbone directly, or can be incorporated in the backbone itself.

For example, a range of hydrolytically degradable amine containing polyesters bearing cationic side chains have been developed (Putnam et al. Macromolecules 32:3658-3662, 1999; Barrera et al. J. Am. Chem. Soc. 115:11010-11011, 1993; Kwon et al. Macromolecules 22:3250-3255, 1989; Lim et al. J. Am. Chem. Soc. 121:5633-5639, 1999; Zhou et al. Macromolecules 23:3399-3406, 1990; each of which is incorporated herein by reference). Examples of these polyesters include poly(L-lactide-co-L-lysine) (Barrera et al. J. Am. Chem. Soc. 115:11010-11011, 1993; incorporated herein by reference), poly(serine ester) (Zhou et al. Macromolecules 23:3399-3406, 1990; which is incorporated herein by reference), poly(4-hydroxy-L-proline ester) (Putnam et al. Macromolecules 32:3658-3662, 1999.; Lim et al. J. Am. Chem. Soc. 121:5633-5639, 1999; each of which is incorporated herein by reference), and more recently, poly[α-(4-aminobutyl)-L-glycolic acid].

In addition, poly(β-amino ester)s, prepared from the conjugate addition of primary or secondary amines to diacrylates, are suitable for use. Typically, poly(β-amino ester)s have one or more tertiary amines in the backbone of the polymer, preferably one or two per repeating backbone unit. Alternatively, a co-polymer may be used in which one of the components is a poly(β-amino ester). Poly(β-amino ester)s are described in U.S. Pat. Nos. 6,998,115 and 7,427,394, entitled “Biodegradable poly(β-amino esters) and uses thereof” and Lynn et al., J. Am. Chem. Soc. 122:10761-10768, 2000, the entire contents of both of which are incorporated herein by reference.

In some embodiments, the polymer can have a formula below:

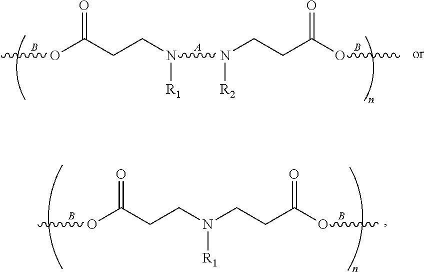

where A and B are linkers which may be any substituted or unsubstituted, branched or unbranched chain of carbon atoms or heteroatoms. The molecular weights of the polymers may range from 1000 g/mol to 20,000 g/mol, preferably from 5000 g/mol to 15,000 g/mol. In certain embodiments, B is an alkyl chain of one to twelve carbons atoms. In other embodiments, B is a heteroaliphatic chain containing a total of one to twelve carbon atoms and heteroatoms. The groups R₁ and R₂ may be any of a wide variety of substituents. In certain embodiments, R₁ and R₂ may contain primary amines, secondary amines, tertiary amines, hydroxyl groups, and alkoxy groups. In certain embodiments, the polymers are amine-terminated; and in other embodiments, the polymers are acrylated terminated. In some embodiments, the groups R₁ and/or R₂ form cyclic structures with the linker A.

Exemplary poly(β-amino esters) include

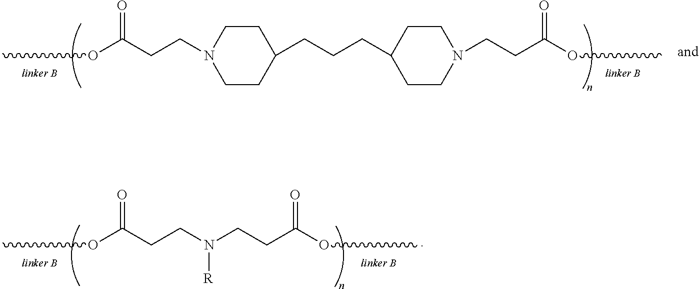

Exemplary R groups include hydrogen, branched and unbranched alkyl, branched and unbranched alkenyl, branched and unbranched alkynyl, aryl, halogen, hydroxyl, alkoxy, carbamoyl, carboxyl ester, carbonyldioxyl, amide, thiohydroxyl, alkylthioether, amino, alkylamino, dialkylamino, trialkylamino, cyano, ureido, a substituted alkanoyl group, cyclic, cyclic aromatic, heterocyclic, and aromatic heterocyclic groups, each of which may be substituted with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups.

Exemplary linker groups B includes carbon chains of 1 to 30 carbon atoms, heteroatom-containing carbon chains of 1 to 30 atoms, and carbon chains and heteroatom-containing carbon chains with at least one substituent selected from the group consisting of branched and unbranched alkyl, branched and unbranched alkenyl, branched and unbranched alkynyl, amino, alkylamino, dialkylamino, trialkylamino, aryl, ureido, heterocyclic, aromatic heterocyclic, cyclic, aromatic cyclic, halogen, hydroxyl, alkoxy, cyano, amide, carbamoyl, carboxylic acid, ester, carbonyl, carbonyldioxyl, alkylthioether, and thiol groups. The polymer may include, for example, between 5 and 10,000 repeat units.

In some embodiments, the poly(β-amino ester)s are selected from the group consisting of

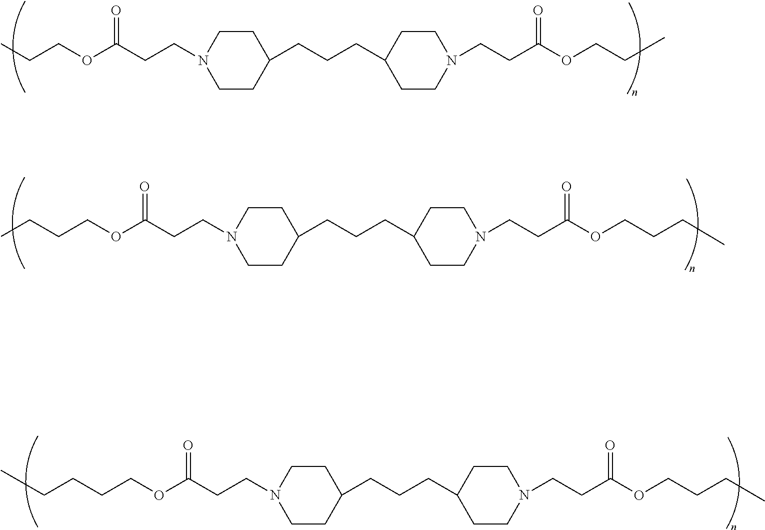

derivatives thereof, and combinations thereof.

Alternatively or additionally, zwitterionic polyelectrolytes may be used. Such polyelectrolytes may have both anionic and cationic groups incorporated into the backbone or covalently attached to the backbone as part of a pendant group. Such polymers may be neutrally charged at one pH, positively charged at another pH, and negatively charged at a third pH. For example, a decomposable film may be constructed by LbL deposition using dip coating in solutions of a first pH at which one layer is anionic and a second layer is cationic. If such a decomposable film is put into a solution having a second different pH, then the first layer may be rendered cationic while the second layer is rendered anionic, thereby changing the charges on those layers.

The composition of degradable polyeletrolyte layers can be fine-tuned to adjust the degradation rate of each layer within the film, which is believe to impact the release rate of drugs. For example, the degradation rate of hydrolytically degradable polyelectrolyte layers can be decreased by associating hydrophobic polymers such as hydrocarbons and lipids with one or more of the layers. Alternatively, polyelectrolyte layers may be rendered more hydrophilic to increase their hydrolytic degradation rate. In certain embodiments, the degradation rate of a given layer can be adjusted by including a mixture of polyelectrolytes that degrade at different rates or under different conditions. In other embodiments, polyanionic and/or polycationic layers may include a mixture of degradable and non-degradable polyelectrolytes. Any non-degradable polyelectrolyte can be used. Exemplary non-degradable polyelectrolytes that could be used in thin films include poly(styrene sulfonate) (SPS), poly(acrylic acid) (PAA), linear poly(ethylene imine) (LPEI), poly(diallyldimethyl ammonium chloride) (PDAC), and poly(allylamine hydrochloride) (PAH).

Alternatively or additionally, the degradation rate may be fine-tuned by associating or mixing non-biodegradable, yet biocompatible polymers (polyionic or non-polyionic) with one or more of the polyanionic and/or polycationic layers. Suitable non-biodegradable, yet biocompatible polymers are well known in the art and include polystyrenes, certain polyesters, non-biodegradable polyurethanes, polyureas, poly(ethylene vinyl acetate), polypropylene, polymethacrylate, polyethylene, polycarbonates, and poly(ethylene oxide)s.

Polymeric Cyclodextrins

Decomposable films provided herein can comprise at least one layer (cationic or anionic layer) that is or comprises a polymeric cyclodextrin. Cyclodextrins can act as carriers for releasable agents intended to be released from such films. In some embodiments, a decomposable film comprising a polymeric cyclodextrin is useful for release of small molecules. Such a decomposable film may be particularly useful in delivering neutral and hydrophobic small molecules with controlled release kinetics, while maintaining their activities (e.g., therapeutic activities).

Cyclodextrins (sometimes called cycloamyloses) are cyclic oligosaccharides containing α-D-glucopyranose units linked by α-1,4 glycosidic bonds. Common types of cyclodextrins include the α-cyclodextrins (comprised of 6 units), β-cyclodextrins (comprised of 7 units), and γ-cyclodextrins (comprised of 8 units). Other types of cyclodextrins include the δ-cyclodextrins (comprised of 9 units) and the ε-cyclodextrins (comprised of 10 units). Cyclodextrins comprising 5 or more than 10 glucopyranose units are also known and/or have been synthesized. For example, large cyclodextrins containing 32 1,4-anhydroglucopyranoside units have been characterized. Large cyclodextrins containing at least 150 glucopyranoside units are also known.

Because of the chair conformation of the glycopyranose units, cyclodextrins are generally toroidally shaped and shaped like a truncated cone. The cavities have different diameters depending on the number of glucose units. For example, the diameters of the cavities of empty cyclodextrin molecules (as measured as the distance between anomeric oxygen atoms) may be approximately for 0.56 nm for α-cyclodextrins, approximately 0.70 nm for β-cyclodextrins, or 0.88 for γ-cyclodextrins.

Decomposable films can have a polymeric cyclodextrin, that is, a polymer comprising a cyclodextrin backbone and/or a cyclodextrin as a pendant group. Cyclodextrins of a variety of types may be used in polymeric form, including α-, β-, and γ-cyclodextrins. Modified cyclodextrins may also be used in polymeric form. For example, cyclodextrin derivatives include, but not limited to, those disclosed in WO 2010/021973, the contents of which are all incorporated herein by reference.

Polymeric cyclodextrins may be synthesized by methods known in the art. (See, e.g., Martin et al. 2006. “Solubility and Kinetic Release Studies of Naproxen and Ibuprofen in Soluble Epichlorohydrin-β-cyclodextrin Polymer,” Supramolecular Chemistry. 18(8): 627-631, the contents of which are herein incorporated by reference in their entirety). Examples of polymeric cyclodextrins include polymers of epi-chlorohydrin-β-cyclodextrin (β-CDEPI), carboxymethyl β-cyclodextrin (BCD), etc.

Polymeric cyclodextrins can be substituted with various groups or moieties, which can alter physical properties, and/or chemical properties of the polymer. For example, solubility in water and/or charges of polymeric cyclodextrins may modified by substituent groups. Substitution can be associated with the polymer backbone or the pendant groups. In some embodiments, cylcodextrin is modified directly. In other embodiments, other portion of the polymer is modified with substituent groups. Variations of cyclodextrins have different solubilities may facilitate delivery of a wide range of agents. The ability to adjust charge type or density can be helpful for LBL film construction.

In some embodiments, the polymer types are crosslinked cyclodextrins. Some of these randomly crosslinked polymers are water soluble; for example, epichlorohydrin-crosslinked β-cyclodextrin has higher aqueous solubility than β-cyclodextrin. Additional exemplary polymeric cyclodextrins are described by Brewster et al. (Brewster et al., Nature Reviews (3), 1023-1035, 2004), which is incorporated herein by reference.

In addition to polymers having cyclodextrin backbone, polymers having cyclodextrins as pendant groups may also be used. These types of polymers can have various polymer backbones and functionalized cyclodextrins. Generally, polymer backbones can have various lengths, molecular weight, charges and substituent groups as described above. Exemplary backbone polymers, including, but not limited to, polyacrylic esters, polyallylamines, polymethacrylates, chitosan, polyester, polyethlenimine, and dendrimers. In some embodiments, a backbone polymer can be degradable polymers as previously described. In certain embodiments, the polymer is a poly(β-amino esters), which is conjugated with cyclodextrins with or without additional linkers and/or functional groups. The number of cyclodextrin per repeat unit in the polymer can also be readily adjust for practical use. For example, the higher density of cyclodextrins in the polymer, the larger loading capacity the polymer theoretically has.

Decomposable films comprising polymeric cyclodextrins generally can be associated with releasable agents that are intended to be released. Associations between cyclodextrins and releasable agents can be formed before film construction. A layer comprising cyclodextrins and releasable agents associated with is then deposited together onto a substrate for constructing a decomposable film in accordance with the present invention.

In some embodiments, cyclodextrins form a complex with a releasable agent. Associations between cyclodextrins and releasable agents are typically loose, and bonding between them is weaker than in a covalent bond. A complex may be an inclusion complex, with a cyclodextrin molecule acting as the “host” molecule. It is also possible for a cyclodextrin to form a non-inclusion complex with a releasable agent.

Polyions

Polyionic layers may be used in film construction and placed next to a layer having an opposite charge. In various embodiments, a decomposable film can comprise one or more polyions. In some embodiments, a polyionic layer is or comprises a polyanion. In some embodiments, a polyionic layer is or comprise a polycation.

For example, in some embodiments, a decomposable film comprise a tetralayer unit having the structure (degradable cationic polyelectrolyte/polyanion/cationic polymeric cyclodextrin/polyanion). (Structures with reversed or modified charge schemes, e.g., comprising anionic polyelectrolytes, polycations, and anionic cyclodextrins, may also be possible.) In some embodiments, a decomposable film comprise a tetralayer unit having the structure (degradable cationic polyelectrolyte/polyanion/cationic drug layer/polyanion). (Structures with reversed or modified charge schemes, may also be possible.)

In some embodiments, polyions are not degradable, though they may be. Polyions used herein are generally biologically derived, though they need not be. Polyions that may be used include charged polysaccharides. In some embodiments, polysaccharides include glycosaminoglycans such as heparin, chondroitin, dermatan, hyaluronic acid, etc. (Some of these terms for glycoasminoglycans are often used interchangeably with the name of a sulfate form, e.g., heparan sulfate, chondroitin sulfate, etc. It is intended that such sulfate forms are included among a list of exemplary polyions used in accordance with the present invention. Similarly, other derivatives or forms of such polysaccharides may be incorporated into films.)

In some embodiments, polyions alter or tune characteristics of a decomposable film that are useful for medical applications. For example, the degradation rate of a decomposable film can be adjusted by combining with a degradable polyeletrolyte as discussed in above section of degradable polyelectrolytes). Polyions may also interact or impart a layer comprising a releasable agent to be released, and thus adjust the release rate/kinetics of the releasable agent. Various polyions as discussed above can be used and exemplary ones demonstrated their effect to the release rate/kinetics in the Examples 2 and 3 below.

### Releasable Agents

According to the present invention, decomposable films can include one or more releasable agents for delivery. In some embodiments, a releasable agent can be associated with individual layers of a decomposable film for incorporation, affording the opportunity for exquisite control of loading and release from the film. In certain embodiments, a releasable agent is incorporated into a decomposable film by serving as a layer.

In theory, any agents including, for example, therapeutic agents (e.g. antibiotics, NSAIDs, glaucoma medications, angiogenesis inhibitors, neuroprotective agents), cytotoxic agents, diagnostic agents (e.g. contrast agents; radionuclides; and fluorescent, luminescent, and magnetic moieties), prophylactic agents (e.g. vaccines), and/or nutraceutical agents (e.g. vitamins, minerals, etc.) may be associated with the decomposable film disclosed herein to be released.

In some embodiments, compositions and methods in accordance with the present invention are particularly useful for release of one or more therapeutic agents. Exemplary agents include, but are not limited to, small molecules (e.g. cytotoxic agents), nucleic acids (e.g., siRNA, RNAi, and microRNA agents), proteins (e.g. antibodies), peptides, lipids, carbohydrates, hormones, metals, radioactive elements and compounds, drugs, vaccines, immunological agents, etc., and/or combinations thereof. In some embodiments, a therapeutic agent to be delivered is an agent useful in combating inflammation and/or infection.

In some embodiments, a therapeutic agent is a small molecule and/or organic compound with pharmaceutical activity. In some embodiments, a therapeutic agent is a clinically-used drug. In some embodiments, a therapeutic agent is or comprises an antibiotic, anti-viral agent, anesthetic, anticoagulant, anti-cancer agent, inhibitor of an enzyme, steroidal agent, anti-inflammatory agent, anti-neoplastic agent, antigen, vaccine, antibody, decongestant, antihypertensive, sedative, birth control agent, progestational agent, anti-cholinergic, analgesic, anti-depressant, anti-psychotic, β-adrenergic blocking agent, diuretic, cardiovascular active agent, vasoactive agent, anti-glaucoma agent, neuroprotectant, angiogenesis inhibitor, etc.

In some embodiments, a therapeutic agent may be a mixture of pharmaceutically active agents. For example, a local anesthetic may be delivered in combination with an anti-inflammatory agent such as a steroid. Local anesthetics may also be administered with vasoactive agents such as epinephrine. To give but another example, an antibiotic may be combined with an inhibitor of the enzyme commonly produced by bacteria to inactivate the antibiotic (e.g., penicillin and clavulanic acid).

In some embodiments, a therapeutic agent may be an antibiotic. Exemplary antibiotics include, but are not limited to, β-lactam antibiotics, macrolides, monobactams, rifamycins, tetracyclines, chloramphenicol, clindamycin, lincomycin, fusidic acid, novobiocin, fosfomycin, fusidate sodium, capreomycin, colistimethate, gramicidin, minocycline, doxycycline, bacitracin, erythromycin, nalidixic acid, vancomycin, and trimethoprim. For example, β-lactam antibiotics can be ampicillin, aziocillin, aztreonam, carbenicillin, cefoperazone, ceftriaxone, cephaloridine, cephalothin, cloxacillin, moxalactam, penicillin G, piperacillin, ticarcillin and any combination thereof.

An antibiotic may be bacteriocidial or bacteriostatic. Other anti-microbial agents may also be used in accordance with the present invention. For example, anti-viral agents, anti-protazoal agents, anti-parasitic agents, etc. may be of use.

In some embodiments, a therapeutic agent may be an anti-inflammatory agent. Anti-inflammatory agents may include corticosteroids (e.g., glucocorticoids), cycloplegics, non-steroidal anti-inflammatory drugs (NSAIDs), immune selective anti-inflammatory derivatives (ImSAIDs), and any combination thereof. Exemplary NSAIDs include, but not limited to, celecoxib (Celebrex®); rofecoxib (Vioxx®), etoricoxib (Arcoxia®), meloxicam (Mobic®), valdecoxib, diclofenac (Voltaren®, Cataflam®), etodolac (Lodine®), sulindac (Clinori®), aspirin, alclofenac, fenclofenac, diflunisal (Dolobid®), benorylate, fosfosal, salicylic acid including acetylsalicylic acid, sodium acetylsalicylic acid, calcium acetylsalicylic acid, and sodium salicylate; ibuprofen (Motrin), ketoprofen, carprofen, fenbufen, flurbiprofen, oxaprozin, suprofen, triaprofenic acid, fenoprofen, indoprofen, piroprofen, flufenamic, mefenamic, meclofenamic, niflumic, salsalate, rolmerin, fentiazac, tilomisole, oxyphenbutazone, phenylbutazone, apazone, feprazone, sudoxicam, isoxicam, tenoxicam, piroxicam (Feldene®), indomethacin (Indocin®), nabumetone (Relafen®), naproxen (Naprosyn®), tolmetin, lumiracoxib, parecoxib, licofelone (ML3000), including pharmaceutically acceptable salts, isomers, enantiomers, derivatives, prodrugs, crystal polymorphs, amorphous modifications, co-crystals and combinations thereof.

Examples of ocular indications requiring treatment with medications include, but are not limited to, postoperative inflammation, iritis, uveitis, keratitis, conjunctivitis, posterior capsular opacification, cystoid macular edema, diabetic retinopathy, diabetic macular edema, macular degeneration, glaucoma and eye trauma.

According to the present invention, any drugs having NSAID-like activity can be used. Suitable compounds having NSAID activity include, but are non-limited to, the non-selective COX inhibitors, selective COX-2 inhibitors, selective COX-1 inhibitors, and COX-LOX inhibitors, as well as pharmaceutically acceptable salts, isomers, enantiomers, polymorphic crystal forms including the amorphous form, co-crystals, derivatives, prodrugs thereof.

Those skilled in the art will recognize that this is an exemplary, not comprehensive, list of agents that can be released using compositions and methods in accordance with the present invention. In addition to a therapeutic agent or alternatively, various other releasable agents may be associated with a decomposable film for controlled release in accordance with the present invention. For example, a releasable agent can be used in effectively preventing unnecessary cell growth on the surface of an implanted IOL. A releasable agent that can inhibit growth of cells or other membrane formation can be associated with a decomposable film. For example, antifibroblastic growth factor may be associated with a decomposable film and released in a controlled manner.

### Substrates

A variety of entities or materials can be used as a substrate for constructing decomposable films. For example, a substrate (e.g., a bodily device) may be coated with one or more decomposable films in accordance with the present invention.

Exemplary entities or materials include, but are not limited to, metals (e.g., gold, silver, platinum, and aluminum); metal-coated materials; metal oxides; plastics; ceramics; silicon; glasses; mica; graphite; hydrogels; and polymers such as polyamides, polyphosphazenes, polypropylfumarates, polyethers, polyacetals, polycyanoacrylates, polyurethanes, polycarbonates, polyanhydrides, polyorthoesters, polyhydroxyacids, polyacrylates, ethylene vinyl acetate polymers and other cellulose acetates, polystyrenes, poly(vinyl chloride), poly(vinyl fluoride), poly(vinyl imidazole), poly(vinyl alcohol), poly(ethylene terephthalate), polyesters, polyureas, polypropylene, polymethacrylate, polyethylene, poly(ethylene oxide)s and chlorosulphonated polyolefins; and combinations thereof. In some embodiments, a substrate may comprise more than one material to form a composite.

Intraocular Lenses (IOLs)

To make a IOL system in accordance with the present invention, one or more decomposable films may be deposited on an IOL to release one or more releasable agents that treat and/or prevent one or more diseases or conditions (such as ocular inflammation, infection, etc.).

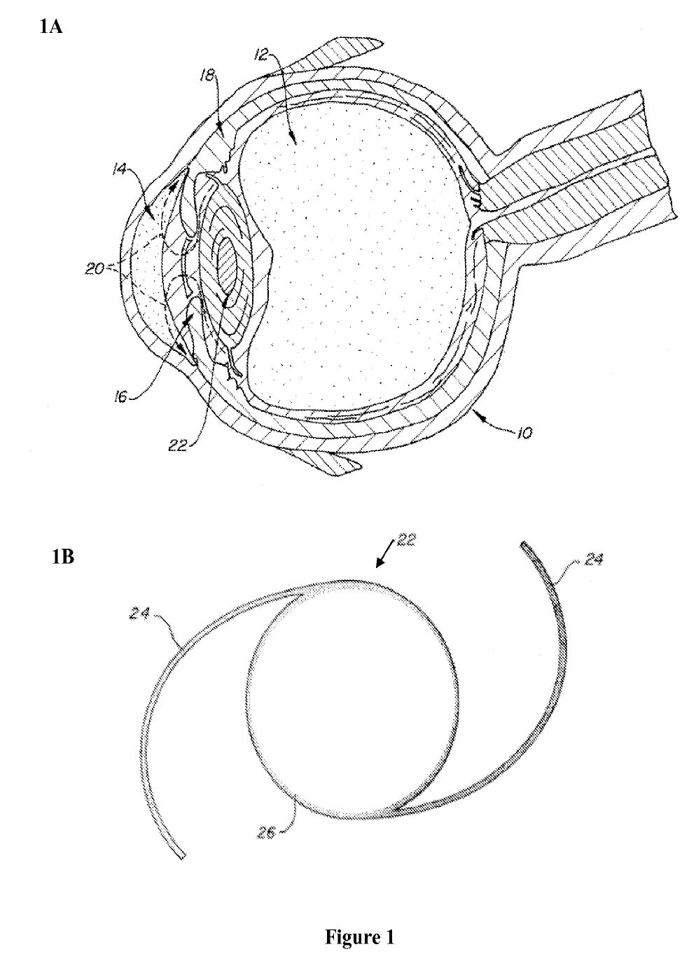

With reference to FIG. 1A, the normal drainage of fluid in an eye 10 is from the back (posterior 12) to front (anterior 14) chamber, with the line of demarcation between the chambers being the iris 16. The normal aqueous fluid of the eye is secreted by the ciliary body 18 located just behind the iris 16 and from there it passes forward through the pupil to reach the anterior chamber 14 (also see description in U.S. Pat. No. 5,554,187, which is incorporated herein by reference). Here the fluid is resorbed into ocular veins through special channels known as Schlemm's canals. This fluid flow is shown by the dashed arrow 20. After cataract extraction surgery, inflammation always occurs to some extent within the anterior chamber 14 of the eye 10. There is also the potential for intra-ocular infection.

In general, an IOL includes an optic and one or more haptics. With reference to FIG. 1B, the IOL 22 is a conventional implantable IOL typically made of a plastic or elastomer material. The optic 26 is secured within the eye in the capsular bag by means of haptics 24. Surgical techniques for implanting the IOL 22 are well known in the art of intra-ocular surgery.

According to the present invention, IOLs herein can include all IOLs, for example, phakic IOLs, bifocal IOLs, multifocal IOLs, standard IOLs, etc. IOLs may be any of a variety of shapes, including opthalmic (convex-concave), biconvex, plano-convex, meniscus, plano-concave, and biconcave.

IOLs may be formed from any acceptable materials known to those skilled in the art such as polumethylmethacrylate (PMMA), silicone, acrylates, hydrogels or any combination thereof. Additionally or alternatively, hydrophobic IOLs can be made of materials including acrylics, acrylates, poly siloxanes, water absorbing acrylates such as polyhydroxyethylmethyacrylate (Poly HEMA), polyvinyl alcohol (PVA), or combinations thereof.

For example, an IOL may be an optical implant for replacement of the human crystalline lens in patients who have cataracts or other lens opacities. It generally is designed to be implanted into the capsular bag following extracapsular cataract extraction or phacoemulsification. An optical portion (i.e., the “optic”) of an IOL is typically comprised of a high refractive index soft acrylic material (acrylate/methacrylate) and this material is capable of being folded prior to insertion allowing placement through a small corneal incision (significantly less than the diameter of the optic and often 2-3 mm or less in size). In such cases, the IOL is placed inside the eye using a specialized insertion instrument and gently unfolded to form a full-size lens body inside the capsular bag. The “haptics” of the lens attach to the optic and form contacts with the capsular bag to stabilize its position once implanted. In some embodiments, haptics are made of the same material as an optic, and in other embodiments they are made of slightly different materials, but also often acrylic. Not all IOLs used in accordance with the present invention are foldable, and such non-foldable IOLs cannot be implanted through a small incision. They require implantion through a large incision at least as large as the diameter of the optic. Such large incisions require sutures for closure whereas small incisions (<2-3 mm) often do not require sutures. The current standard of care for routine cataract surgery is the use of foldable IOLs, because the small incisions are less traumatic to the ocular surface and can be performed without sutures. Such foldable IOLs are suitable for use in accordance with the present invention.

In various embodiments, a commercial foldable IOL can be used in accordance with the present invention. For example, the AVS, Inc. XACT® Foldable Hydrophobic UV Light-Absorbing Posterior Chamber IOL, is a three-piece IOL with a biconvex optic made from a proprietary high refractive index soft acrylic material, allowing the device to be folded and inserted though an incision smaller than of the optic. The supporting haptics are made from polyvinylidene fluoride (PVDF) monofilament. An another exemplary foldable IOL that may be suitable for use in accordance with the present invention, is ACRYSOF® Acrylic Foldable UV-Absorbing Multipiece Posterior Chamber Lenses. A document with detailed product information of the foldable IOL is attached hereto as Appendix A, and the contents of which are incorporated herein by reference.

### Assembly and Coating Methods

There are several advantages to LBL assembly techniques used in accordance with the present invention, including mild aqueous processing conditions (which may allow preservation of biomolecule function); nanometer-scale conformal coating of surfaces; and the flexibility to coat objects of any size, shape or surface chemistry, leading to versatility in design options. According to the present invention, one or more decomposable films can be assembled and/or deposited on a substrate using a LBL technique. The coating compositions and methods provided herein may be used for coating a substrate (e.g., bodily devices such as an IOL). In various embodiments, one or more decomposable films can be the same. In some embodiments, one or more decomposable films can be different in film materials (e.g., polymers), film architecture (e.g., bilayers, tetralayer, etc.), film thickness, and/or agent association.

It will be appreciated that an inherently charged surface of a substrate can facilitate LbL assembly of a decomposable film on the substrate. In addition, a range of methods are known in the art that can be used to charge the surface of a substrate, including but not limited to plasma processing, corona processing, flame processing, and chemical processing, e.g., etching, micro-contact printing, and chemical modification.

Additionally or alternatively, substrates can be primed with specific polyelectrolyte bilayers such as, but not limited to, LPEIISPS, PDAC/SPS, PAH/SPS, LPEI/PAA, PDAC/PAA, and PAH/PAA bilayers, that form readily on weakly charged surfaces and occasionally on neutral surfaces. Exemplary polymers can be used as a primer layer include poly(styrene sulfonate) and poly(acrylic acid) and a polymer selected from linear poly(ethylene imine), poly(diallyl dimethyl ammonium chloride), and poly(allylamine hydrochloride). It will be appreciated that primer layers provide a uniform surface layer for further LBL assembly and are therefore particularly well suited to applications that require the deposition of a uniform thin film on a substrate that includes a range of materials on its surface, e.g., an implant or a complex tissue engineering construct.

In some embodiments, the LbL assembly of a decomposable film may involve a series of dip coating steps in which a substrate is dipped in alternating polycationic and polyanionic solutions. Additionally or alternatively, it will be appreciated that deposition of alternating polycationic and polyanionic layers may also be achieved by spray coating, dip coating, brush coating, roll coating, spin casting, or combinations of any of these techniques.

In some embodiments, coating a substrate with a decomposable film may involve masking to facilitate multi-region coating. A physical mask, a chemical mask or combination thereof can be used. For example, materials of a physical mask can be paper, wood, metal or plastic or combination thereof. In some embodiments, a physical mask does not contact the substrate to be coated. As for chemical masking, materials can be a water soluble coating, a lipid soluble coating or combination thereof. In certain embodiments, a water soluble coating is a polysaccharide. In certain embodiments, a lipid soluble coating may be wax, adhesive, silicone, methacrylic polymers, or combination thereof.

Methods disclosed herein may be used to create three-dimensional microstructures. For example, a decomposable film may be deposited on a substrate that can be dissolved to leave a hollow shell of the film. Alternatively or additionally, multi-layers may be deposited on substrates having regions that are more and less degradable. Degradation of the degradable portions leaves a three-dimensional microstructure. In a first step, the surface of a substrate is divided into regions in which LbL deposition of an inventive decomposable film is more or less favorable. In one embodiment, a pattern of self-assembled monolayers (SAMs) is deposited on a substrate surface by microcontact printing (see, for example, U.S. Pat. No. 5,512,131 to Kumar et al., see also Kumar et al., Langmuir 10:1498, 1994; Jiang and Hammond, Langmuir, 16:8501, 2000; Clark et al., Supramolecular Science 4:141, 1997; and Hammond and Whitesides, Macromolecules 28:7569, 1995). In some embodiments, the substrate surface is neutral and the exposed surface of the deposited SAMs is polar or ionic (i.e., charged). A variety of polymers with polar or ionic head groups are known in the art of self-assembled monolayers. In some embodiments, a uniform coating of a polymer is deposited on a substrate, and that coating is transformed into a patterned layer by means of photolithography. Other embodiments are also contemplated in which the substrate surface is selectively exposed to plasmas, various forms of electromagnetic radiation, or to electron beams.

In yet other embodiments, the substrate may possess the desired surface characteristics by virtue of its inherent composition. For example, the substrate may be a composite in which different regions of the surface have differing compositions, and thus different affinities for the polyelectrolyte to be deposited. In a second step, polyelectrolyte layers of alternating charge are deposited by LbL on receptive regions of the surface as described for a homogeneous surface above and for selective regions as described in Jiang and Hammond, Langmuir, 16:8501, 2000; Clark et al., Supramolecular Science 4:141, 1997; and Hammond and Whitesides, Macromolecules 28:7569, 1995. The surface is subsequently flooded with a non-degradable polymer and placed in a medium wherein at least a portion of the polyelectrolyte layers degrade, thereby creating a three-dimensional “tunnel-like” structure that reflects the pattern on the original surface. It will be appreciated that more complex microstructures could be created based on these simple principles (e.g., by depositing SAMs with different electrostatic character in different regions of a substrate surface and/or by iterative additions of subsequent structures above the deposited non-degradable polymer).

According to the present invention, decomposable films can be deposited on an IOL. One or more decomposable films may be deposited on an entire IOL or one or more portions of an IOL. In some embodiments, an optic, one of more haptics, or any combinations thereof can be selectively coated with decomposable films. In some embodiments, one of more decomposable films can be used to coat one or more portions of the posterior surface, one or more portions of the anterior surface, one or more circumferential edges of an IOL, or any combinations thereof. In certain embodiments, a decomposable film may be deposited away from the visual axis of an IOL such as by placing it near the periphery of the IOL. In these embodiments, the decomposable film do not interfere with vision.

### Use and Applications

Compositions and methods provide herein can be of use various application such as coating bodily devices (e.g., medical devices) using a multi-layer decomposable film assembled LBL.

For example, deposited on an IOL is one or more decomposable films in accordance with the present invention. Such an IOL system comprising an IOL coated with a decomposable film can be used with conventional surgical procedures. Exemplary methods and apparatus for a foldable IOL are described in U.S. Pat. No. 4,785,810, which is incorporated herein by reference.

The compositions and methods provided herein may be particularly useful in combating inflammation and infection after eye surgery (e.g., after implantation of an IOL in cataract surgery) or concomitant eye conditions requiring treatment with medications (e.g., glaucoma, diabetic retinopathy, macular degeneration, dry eye disease, ocular allergy). At least some advantages of inventive compositions and methods disclosed herein are that decomposable films may not substantively alter/modify the properties of an IOL, and may not make surgical introduction of the IOL any more difficult than with a conventional IOL. It is also contemplated that an IOL coated with a decomposable film in accordance with the present invention may facilitate IOL implantation and may demonstrate improved animal or clinical data.

Also provided in the disclosure are methods of releasing one or more releasable agents from a decomposable film. Such methods generally comprise steps of providing a decomposable film and placing the film in a medium in which at least a portion of the film decomposes via the substantially sequential removal of at least a portion of the layers having the first charge and degradation of layers having the second charge. A medium can be, for example, provided from in vivo environment such as a subject's body (e.g., for implants such as an IOL). In some embodiments, a medium can be provided in an artificial environment (e.g., for tissue engineering scaffolds). Buffers such as phosphate-buffered saline may also serve as a suitable medium.

Certain characteristics of a degradable thin film-coated substrate may be modulated to achieve desired doses of releasable agents and/or release kinetics. Doses may be modulated, for example, by changing the number of multilayer units that make up the film, the type of degradable polyelectrolyte used, the type of polyion (if any) used, and/or concentrations of solutions of releasable agents used during construction of the films. Similarly, release kinetics (both rate of release and duration of release of an agent) may be modulated by changing any or a combination of the aforementioned factors.

In some embodiments, the dose of a releasable agent incorporated in a decomposable film for release can be about or greater than 1 mg/cm². In some embodiments, the dose of a releasable agent incorporated in a decomposable film can be about or less than 100 μg/cm². In some embodiments, the dose of a releasable agent incorporated in a decomposable film can be about or less than 50 μg/cm². In some embodiments, the dose of a releasable agent incorporated in a decomposable film can be about 10 mg/cm², about 1 mg/cm², 500 μg/cm², about 200 μg/cm², about 100 μg/cm², about 50 μg/cm², about 40 μg/cm², about 30 μg/cm², about 20 μg/cm², about 10 μg/cm², or about 5 μg/cm². In some embodiments, the dose of a releasable agent incorporated in a decomposable film can be in a range of any two values above.

Release of a releasable agent may follow linear kinetics over a period of time. Release of multiple drugs from a decomposable film may be complicated by interactions between layers, and/or drugs. Such a release profile may be desirable to effect a particular dosing regimen. During all or a part of the time period of release, release may follow approximately linear kinetics.

Some embodiments provide systems for releasing a releasable agent over a period of at least about 2 days, about 5 days, about 10 days, about 12 days, about 20 days, about 30 days, 50 or about 100 days. In some embodiments, a releasable agent can be released in a controlled manner over a period of any two values above.

### EXAMPLES

### Example 1

### Construction of Layer-by-Layer Films with Multiple Drugs

Characteristics of layer-by-layer (LbL) films (such as, film stability, release kinetics of drugs, etc.) vary depending on materials used to construct the films. In this Example, exemplary tetralayer architectures with alternating layers of polyanions and polycations were constructed layer-by-layer using different deposition methods (e.g., dipping or spraying). As model drugs, vancomycin (as an antibiotic) and diclofenac (as a non-steroidal anti-inflammatory drug (NSAID)) were incorporated for drug release.

Materials and Reagents

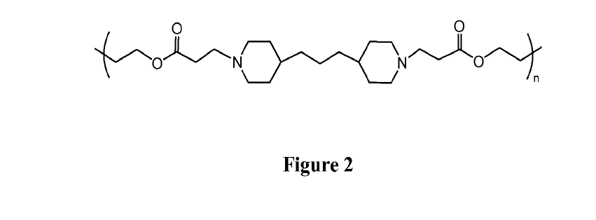

Poly (β-amino ester)s (e.g., Poly 2 as illustrated in FIG. 2) were synthesized as previously described (Lynn et al. 2000. Journal of the American Chemical Society. 122: 10761-10768.). Vancomycin, alginate (Mₙ=120-190 kDa), poly(sodium 4-styrene-sulfonate) (SPS, Mₙ=70 kDa), and sodium acetate buffer (3 M) were purchased from Sigma-Aldrich (St. Louis, Mo.). Diclofenac and polyCD (2.8% substituted) were purchased from TCI America (Portland, Oreg.) and CTD, Inc. (Gainesville, Fla.), respectively. Chondroitin sulfate sodium salt (Mₙ=85 kDa) was purchased from TCI International (Tokyo, Japan). Dextran sulfate sodium salt (Mₙ=500 kDa) and linear polyethyleneimine (LPEI, Mₙ=25 kDa) were purchased from Polysciences (Warrington, Pa.). Silicon and glass substrates were obtained from Silicon Quest International (Santa Clara, Calif.) and VWR Scientific (Edison, N.J.), respectively. Intraocular lenses were generously donated by Aurolab (Aravind Eye Care System, Madurai, India). Vicryl sutures and latex-free absorbent sterile pad bandages were obtained from the Department of Comparative Medicine (Massachusetts Institute of Technology) and RiteAid Pharmacy (Harrisburg, Pa.), respectively. Dulbecco's phosphate buffered saline (PBS, 0.1 M) was purchased from Invitrogen (Carlsbad, Calif.). Deionized water (18.2 MΩ, Milli-Q Ultrapure Water System, Millipore) was utilized in all experiments. S. aureus 25923 was obtained from ATCC (Manassas, Va.). Cation-adjusted Mueller Hinton broth (CaMHB), Bacto agar, and vancomycin susceptibility test disks were obtained from BD Biosciences (San Jose, Calif.). Cyclooxygenase fluorescence inhibitor screening assay kit was purchased from Cayman Chemical (Charlotte, N.C.).

Film Assembly

Prior to assembly, substrates (approximately 1 cm²) were cleaned, plasma etched, and coated with (LPEI/SPS)₁₀ base layers as previously described (A. Shukla et al., Small, 6 (2010) 2392-2404). Composite films containing both diclofenac and vancomycin were created by combining single-therapeutic film architectures whose assembly has been previously described R. C. Smith et al., Angew. Chemie Int. Edit., 48 (2009) 8974-8977). Briefly, antibiotic-only films were built with a tetralayer architecture, denoted: (poly 2/polyanion/vancomycin/polyanion)₆₀, where the polyanion was alginate, chondroitin sulfate, or dextran sulfate and sixty represents the number of tetralayers deposited. All deposition solutions for the antibiotic films were formulated at 2 mg/mL in 0.1 M sodium acetate buffer (pH 5). In dipped LbL films, poly 2 and vancomycin were deposited for 10 minutes, and the polyanions for 7.5 minutes, with 10, 20, and 30 second rinses following each step. For alginate and chondroitin sulfate films, deionized water (pH 5) was used for the rinse steps, and for dextran sulfate films, 0.1 M sodium acetate buffer (pH 5) was used. NSAID-only films were built with bilayer architecture, (poly 2/polyCD-diclofenac)₂₀. The NSAID film poly 2 deposition solution was formulated at 2 mg/mL in 0.1 M sodium acetate buffer (pH 6), while polyCD-diclofenac solution was prepared at 20 mg/mL polyCD and 1.4 mg/mL diclofenac in 0.1 M sodium acetate buffer (pH 6). In short, dipped NSAID film deposition steps lasted 10 minutes, followed by 10, 20, and 30 second rinses in deionized water (pH 6).

Spray LbL films were created using a programmable spray apparatus (Svaya Nanotechnologies). All drug and polyelectrolyte spray deposition steps were 2 seconds, while a single 3 second rinse step was used following each deposition with a flow rate of 0.25 mL/s. All solution formulations used for spray LbL were the same as those used in dipping.

For composite dipped and sprayed films, the NSAID film was either layered directly on a preformed antibiotic film or the NSAID film coated substrate was used for subsequent deposition of antibiotic films. Composite films were also created on intraocular lenses (using dipped LbL), sutures, and bandages (using spray LbL and applying a 50 psi vacuum to the back of the substrate). These materials were pre-treated in the same way as the silicon and glass substrates prior to film assembly.

For all optimal film architectures constructed in this study, film thickness on glass or silicon substrates was monitored using either a spectroscopic ellipsometer (J. A. Woollam Co., Inc. M-2000D) or a surface profilometer (KLA Tencor P-16). For profilometer measurements, films were scored with a razor, tracked over a 700 μm length, and average film thickness was obtained. Device coatings were also examined using a scanning electron microscope (JEOL JSM-6060).

### Example 2

### Film Assembly and Drug Release Characteristics

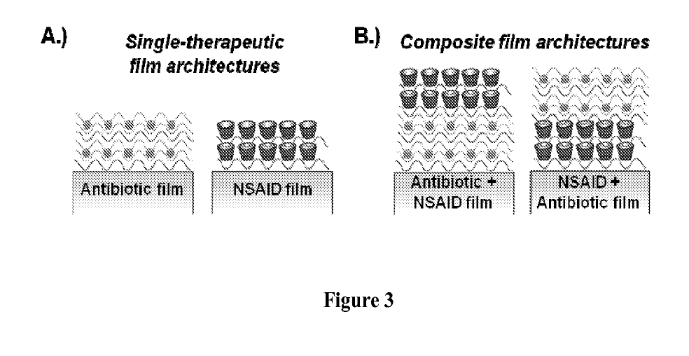

Exemplary film architectures were investigated to formulate dual drug-release films and are shown in FIG. 3. In this Example, single-therapeutic films were studied and such studies were used to facilitate formulating composite films. Composite films using exemplary deposition methods as described in Example 1 were constructed and characterized. All experiments conducted in this work were done in triplicate at minimum. Data is reported as mean±standard deviation. All thickness measurements were taken at a minimum of three locations per sample.

Drug Release

Reagents and solutions were obtained and prepared as described in Example 1. Films were dried under nitrogen after assembly and released in 500 μL of 0.01 M PBS at 37° C. At predetermined time points films were removed and added to fresh PBS aliquots. Vancomycin and diclofenac presence in each of the release samples was quantified with high performance liquid chromatography (Agilent Technologies HPLC, 1100 series) using a C18 reverse phase column (Supelco) equipped with a fluorescence detector. An excitation wavelength of 280 nm and emission wavelength of 355 nm was utilized. Vancomycin fluorescence was monitored with a 70/30 0.01 M PBS/methanol mobile phase, while diclofenac fluorescence was monitored with a 70/30 0.01 M PBS/acetonitrile mobile phase. A flow rate of 1 mL/min and injection volume of 500 μL and 100 μL was used for vancomycin and diclofenac, respectively.

Studying Film Component Interactions

Molecular interactions between film components were examined chromatographically. Interactions between polyCD and vancomycin were studied by dissolving vancomycin at a concentration of 41 μM in polyCD (0, 2, 4, 8, and 16 mM) at pH 5 and 6 in sodium acetate buffer (0.1 M) and sodium chloride (1 M) and examining vancomycin fluorescence with HPLC as described under Drug Release. Interactions between diclofenac and vancomycin were studied by suspending excess diclofenac (34 mM) in vancomycin solutions (1.3 mM, 0.65 mM, and 1.3 μM) in the same four solution conditions and exploring diclofenac solubility (proportional to diclofenac fluorescence) via HPLC after filtering these solutions through 0.2 μm filters.

To quantify diffusion and exchange capabilities of single-therapeutic films, the NSAID-only or antibiotic-only film architectures were introduced to film deposition and wash solutions (described under Film Assembly) for the complementary film for 10 minutes (the maximum deposition time). Following this, each film was rinsed briefly in deionized water to remove non-specifically bound material. It was chromatographically determined how much of the deposition component diffused into the film (by taking these films after treatment and allowing them to release completely in 0.01 M PBS solution and examining these with HPLC) as well as how much of the film therapeutic was displaced in this process (by examining the test solutions with HPLC). A representative antibiotic film architecture containing chondroitin sulfate was used in all of these experiments. A twenty bilayer film assembled analogous to the NSAID-only film but containing no diclofenac, (PolyCD₂₀), was also included in these studies.

Studying Film Component Interactions

Vancomycin activity was assessed using both a modified Kirby-Bauer and microdilution assay. For these assays, S. aureus 25923 in its exponential growth phase was utilized. In the Kirby-Bauer assay, S. aureus at 10⁸ CFU/mL concentration was applied evenly to an agar plate. Film coated bandages, an uncoated control, and a 30 μg vancomycin susceptibility disk were each applied to the coated agar and incubated for 16-18 hours at 37° C., after which the zone of inhibition surrounding the test materials was examined. In the microdilution assay, film released solutions and controls of 0.01 M PBS were serial diluted in CaMHB in a 96 well clear bottom plate. S. aureus was added to each of the film release dilutions and positive controls at a final concentration of 10⁵ CFU/mL, with no bacteria added to the negative controls. After 16-18 hours of incubation with shaking at 37° C., the optical density of each well at 600 nm (proportional to bacteria concentration) was read on a BioTek PowerWave XS plate reader. Normalized bacteria density was calculated.

To quantify diclofenac activity, a COX inhibition assay was utilized. When uninhibited, COX leads to the production of hydroperoxy endoperoxide (PGG₂) from arachadonic acid. PGG₂ reacts with 10-acetyl-3,7-dihydroxyphenoxazine (ADHP) to produce fluorescent resorufin. Resorufin fluorescence upon exposure to film release solution and controls of polyCD, polyCD-diclofenac, and vancomycin solution, was quantified.

### Results

In this study, changes in fluorescence intensity of vancomycin and diclofenac in these mixtures compared to pure drug solutions indicated the formation of complexes between interacting species. Interactions between film components were probed at four different conditions, 0.1 M sodium acetate buffer and 1 M sodium chloride at pH 5 and 6. The 0.1 M pH 5 and 6 solvents represent the previously determined optimal deposition conditions for the vancomycin and diclofenac films, respectively. Two critical interactions were discovered to exist, namely the interaction of polyCD with vancomycin and the interaction of vancomycin with diclofenac.

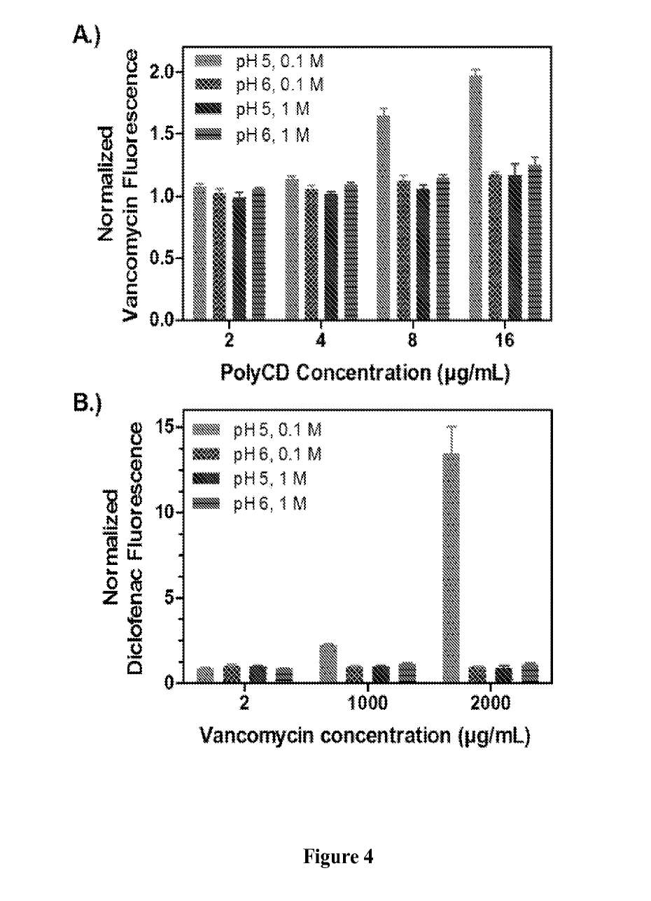

FIG. 4A shows vancomycin fluorescence for a constant vancomycin concentration (34.5 μM) dissolved in varying polyCD concentrations at each solvent condition tested normalized by its fluorescence in pure vancomycin solution (absent any polyCD). Normalized vancomycin fluorescence increased with increasing polyCD concentrations only in the pH 5 (0.1 M) solvent, an indication of an interaction occurring between vancomycin and polyCD at these conditions. At pH 5, vancomycin has a net positive charge of 1, and the cationic vancomycin can interact electrostatically with the anionic polyCD. At pH 6, vancomycin charge is greatly reduced with its isoelectric point near neutral pH, and therefore, this interaction is not promoted at these conditions. Further evidence that this interaction is primarily electrostatic was obtained from results of solutions formulated at the higher ionic strength of 1 M (pH 5). At these conditions, charge screening inhibits the electrostatic polyCD-vancomycin interaction and these solutions no longer show increased normalized vancomycin fluorescence in the presence of polyCD.

Next, we examined mixtures of diclofenac and vancomycin. The hydrophobic diclofenac was suspended in excess in the same four solvent conditions described earlier containing three separate vancomycin concentrations (1.3 μM, 0.65 mM, and 1.3 mM). Note that the 1.3 mM vancomycin concentration represents the concentration of vancomycin used in antibiotic-only film construction. These solutions were filtered to remove non-soluble diclofenac and normalized diclofenac fluorescence was determined by comparing diclofenac fluorescence in these filtered solutions to those of pure filtered diclofenac (absent any vancomycin). The results of this interaction study are summarized in FIG. 4B. At pH 5 in 0.1 M buffer, increasing vancomycin concentration led to increased diclofenac solubilization (directly proportional to diclofenac fluorescence). This effect was nonlinear with no increase in diclofenac solubilization at the lowest vancomycin concentration tested, and an approximate 14 times increase in diclofenac solubility at the highest vancomycin concentration equal to that used in antibiotic-only film assembly. This interaction does not occur at pH 6 conditions (due to reduced vancomycin charge) and at high salt concentration (due to charge screening) suggesting that like the interaction of polyCD and vancomycin, the interaction of diclofenac and vancomycin is primarily electrostatic between the positively charged vancomycin (at pH 5) and the negative charge of the diclofenac carboxyl group.

Based on the discovery of these two interactions, namely the interaction of polyCD and vancomycin as well as the interaction of diclofenac and vancomycin, one can anticipate the behavior of composite films in which the antibiotic component would be deposited upon a preformed NSAID film. Without being bound to any particular theory, it is contemplated that these films would experience increased vancomycin loading compared to antibiotic-only films, due to the electrostatic attraction between polyCD and vancomycin. Additionally, submerging an NSAID film into the vancomycin deposition solution should lead to diclofenac stripping from the existing NSAID film, due to electrostatic attraction of vancomycin and diclofenac.

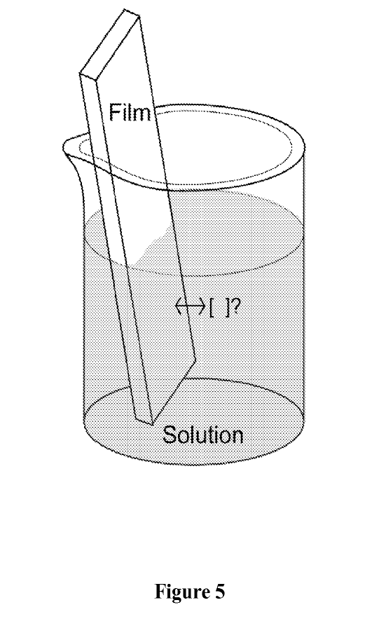

To translate these solution-based observations to films, a series of experiments were conducted to quantify the ability of components to diffuse into and out of single-therapeutic films upon exposure to LbL assembly deposition and wash conditions for the complementary drug containing film. The observed phenomena is related to commonly observed diffusion and exchange behavior in LbL films and found to be strongly dependent on charge density, molecular weight, and ionic strength of the species involved. A schematic of these studies is shown in FIG. 5, while the typical results of this study are summarized in Table 1.

TABLE 1
Diffusion and exchange behavior in single-therapeutic films.

| | | Vancomycin | | Diclofenac | |
|---|---|---|---|---|---|
| | Deposition/ | (μg) | | (μg) | |
| Film | wash solution | In | Out | In | Out |
| (NSAID film)₂₀ | Vancomycin | 5 ± 3 | NA | NA | 4 ± 1 |
| | 0.1M sodium | NA | | | 0 |
| | acetate buffer | | | | |
| | (pH 5) | | | | |
| (PolyCD film)₂₀ | Vancomycin | 13 ± 6 | NA | NA | NA |
| (Antibiotic film)₆₀ | PolyCD- | NA | 4 ± 1 | 28 ± 10 | NA |
| | diclofenac | | | | |
| | PolyCD | | 8 ± 1 | NA | |
| | 0.1M sodium | | 11 ± 3 | | |
| | acetate buffer | | | | |
| | (pH 6) | | | | |

As expected based on the solution interaction studies, NSAID films incorporated large amounts of vancomycin. Additionally, films assembled with polyCD containing no NSAID incorporated larger amounts of vancomycin than those films in which diclofenac was encapsulated in the polyCD, suggesting that the interaction of polyCD and vancomycin is more likely to occur when there is nothing populating the hydrophobic core of the cyclodextrins. Although vancomycin is too large and hydrophilic to completely fit within the polyCD core, the hydrophobic phenolic groups of vancomycin may partially associate with empty cores. An unexpected finding was adsorption of significant diclofenac quantities (approximately 6 times the final loading of an NSAID film) into antibiotic films at the pH 6 deposition conditions of polyCD-diclofenac. This interaction at pH 6 was not visible in solution, although a strong interaction of polyCD and vancomycin as well as diclofenac and vancomycin was observed at pH 5. At pH 6, vancomycin is slightly charged; due to the localized concentration of vancomycin in the antibiotic film versus a dilute solution, it is likely that interactions observed strongly at pH 5 in solution are also visible at pH 6 in the case of the film. Based on these findings, we predicted that films in which the NSAID component is deposited on the antibiotic film would experience increased diclofenac loading.

Each single-therapeutic film was stable in its own wash condition. However, at the pH 6 NSAID film wash conditions, 11±3 μg of vancomycin was lost in the duration of a single deposition step; 64% less vancomycin was lost in the polyCD-diclofenac solution at pH 6. It could be expected that the antibiotic film would be severely destabilized at the pH 6 conditions necessary for NSAID film construction. Without being bound to any particular theory, the level of destabilization is believed to be strongly dependent on the initial stability of the antibiotic-only film, which is heavily dependent on the polyanion choice and deposition technique (spray versus dip LbL). NSAID films were found to be stable at the pH 5 wash conditions of the antibiotic film, retaining all diclofenac. However, as expected from solution interaction studies, in the presence of vancomycin, diclofenac was stripped from these films at significant amounts (comparable to the total loading of an NSAID film). These findings indicated that deposition of antibiotic films upon preformed NSAID films may lead to severely depleted diclofenac loadings and increased vancomycin loadings in these films. The solution based interaction studies appropriately predicted the behavior of single-therapeutic films, and were used to help formulate composite films.

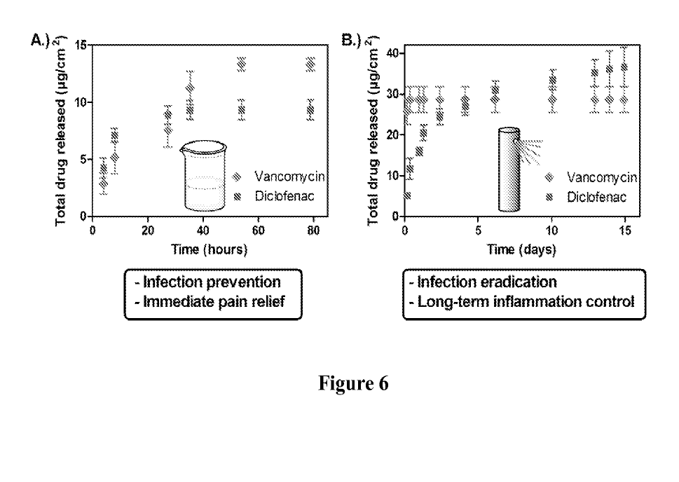

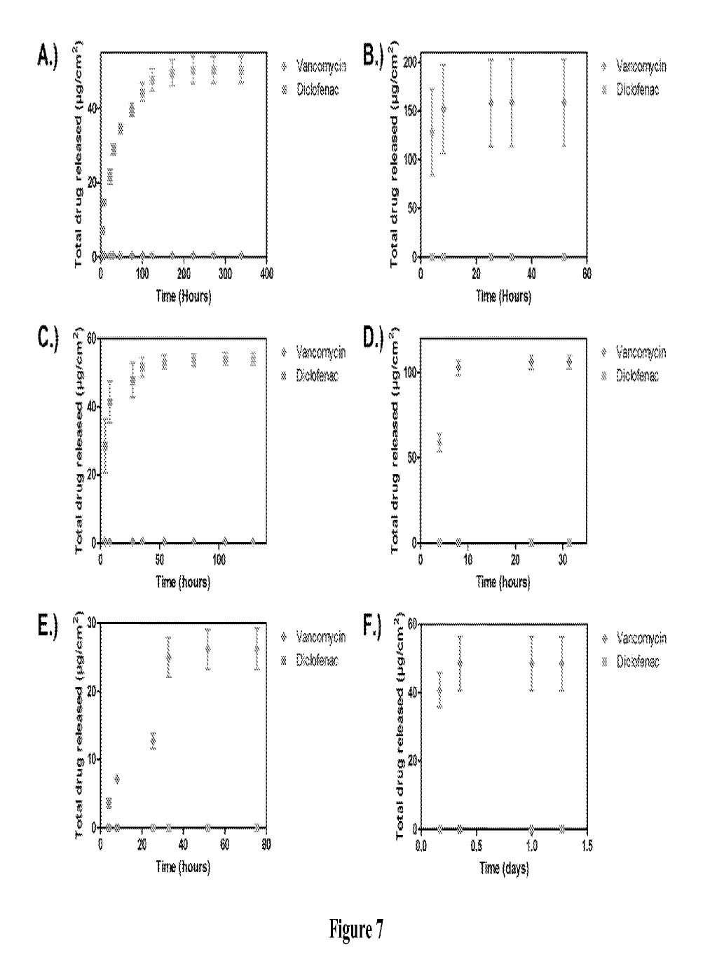

Following extensive interaction studies, we designed composite films using both dip and spray assembly to test our findings and create several optimal multi-drug release film architectures. Table 2 summarizes typical relevant drug loading and release characteristics of these composite films as well as single-therapeutic films (corresponding co-release profiles are exhibited in FIG. 6 or in FIG. 7). In films where the NSAID component was deposited first followed by the antibiotic component, there was increased vancomycin loading as compared to antibiotic-only films. The difference in loading (approximately 1.2 to 1.5 times) is not strongly dependent on the polyanion used in the antibiotic film tetralayer. The spray assembled composite architecture incorporated approximately 1.7 times more vancomycin than a sprayed antibiotic-only film. In addition, diclofenac had been completely stripped from these films. These findings for the composite architecture formulated from an antibiotic film deposited upon an existing NSAID film were all in agreement with the pre-construction interaction studies.

TABLE 2
Total drug loading and release timescale of single-therapeutic and composite films.

| | | | | | | Total |
|---|---|---|---|---|---|---|
| | | | Total | Total | Total | diclofenac |
| | | LbL | vancomycin | vancomycin | diclofenac | release |
| Film | | deposition | loading | release time | loading | time |
| architecture | Polyanion | technique | [μg/cm²] | [days] | [μg/cm²] | [days] |
| (Antibiotic | Alginate | Dip | 89.6 ± 0.3 | 0.3 | NA | NA |
| film)₆₀ | | | | | | |
| (Antibiotic | Chondroitin | Dip | 107.6 ± 0.2 | 2.1 | NA | NA |
| film)₆₀ | sulfate | | | | | |
| (Antibiotic | Dextran | Dip | 21.5 ± 1.5 | 2.2 | NA | NA |
| film)₆₀ | sulfate | | | | | |
| (Antibiotic | Chondroitin | Spray | 28.5 ± 3.0 | 0.3 | NA | NA |
| film)₆₀ | sulfate | | | | | |
| (NSAID | NA | Dip | NA | NA | 5.0 ± 1.0 | 20.0 |
| film)₂₀ | | | | | | |
| (NSAID | NA | Spray | NA | NA | 7.0 ± 0.2 | 7.0 |
| film)₂₀ | | | | | | |
| (Antibiotic | Alginate | Dip | 0.5 ± 0.2 | 1.1 | 54.1 ± 1.9 | 4.4 |
| film)₆₀ + | | | | | | |
| (NSAID | | | | | | |
| film)₂₀ | | | | | | |
| (Antibiotic | Chondroitin | Dip | 0.5 ± 0.3 | 9.3 | 50.3 ± 3.6 | 9.3 |
| film)₆₀ + | sulfate | | | | | |
| (NSAID | | | | | | |
| film)₂₀ | | | | | | |
| (Antibiotic | Dextran | Dip | 13.3 ± 0.5 | 2.3 | 9.4 ± 0.9 | 1.7 |
| film)₆₀ ⁺ | sulfate | | | | | |
| (NSAID | | | | | | |
| film)₂₀ | | | | | | |
| (Antibiotic | Chondroitin | Spray | 28.7 ± 3.2 | 0.4 | 36.2 ± 4.4 | 13.9 |
| film)₆₀ + | sulfate | | | | | |
| (NSAID | | | | | | |
| film)₂₀ | | | | | | |
| (NSAID | Alginate | Dip | 106.1 ± 4.0 | 1.0 | 0 | 0 |
| film)₂₀ + | | | | | | |
| (Antibiotic | | | | | | |
| film)₆₀ | | | | | | |
| (NSAID | Chondroitin | Dip | 158.6 ± 44.8 | 1.4 | 0 | 0 |
| film)₂₀ + | sulfate | | | | | |
| (Antibiotic | | | | | | |
| film)₆₀ | | | | | | |
| (NSAID | Dextran | Dip | 26.1 ± 2.9 | 2.2 | 0 | 0 |
| film)₂₀ + | sulfate | | | | | |
| (Antibiotic | | | | | | |
| film)₆₀ | | | | | | |
| (NSAID | Chondroitin | Spray | 48.5 ± 7.9 | 0.4 | 0 | 0 |
| film)20 + | sulfate | | | | | |
| (Antibiotic | | | | | | |
| film)60 | | | | | | |

In some experiments, NSAID films were deposited on top of preformed antibiotic films to ensure retention of diclofenac in composite films. However, in the case of alginate and chondroitin sulfate dipped films with this architecture, there was little vancomycin retained after NSAID deposition (as predicted by the pH 6 destabilization of vancomycin films). FIG. 6A shows the release profile of the NSAID film built on the more stable dextran sulfate containing vancomycin architecture. This composite film was found to have a thickness of 4.36±0.28 μm, greater than a dextran sulfate dipped antibiotic-only film (thickness of 3.14±0.24 μm). The increased affinity for polyCD-diclofenac in the pre-deposited antibiotic film is expected to lead to interdiffusion and higher loading of polyCD-diclofenac, thereby increasing film thickness (note that dipped NSAID-only films have a thickness of approximately 20 nm). There was an approximate 38% reduction in the incorporated vancomycin in these films during NSAID film deposition. However, the remaining 13.3±0.5 μg/cm² of vancomycin in this film remains highly therapeutic, able to meet and exceed the minimum inhibitory concentration of vancomycin against S. aureus (0.5-2 μg/mL). Additionally, the timescale of vancomycin release from this film architecture was comparable to the antibiotic-only film, approximately 2.3 days, with a linear release profile following the first 4 hours (R²=0.95). This architecture also incorporated approximately 1.9 times more diclofenac than an NSAID-only film (9.4±0.9 μg/cm² versus 5.0±1.0 μg/cm²), also predicted by the interaction studies; release timescale was reduced from 20 to 1.7 days, dictated by the underlying antibiotic film architecture. Approximately 50% of diclofenac was released in the first 4 hours. This architecture led to moderate release times for both drugs at therapeutic doses, appropriate for infection prevention and immediate pain management following injury or surgery, avoiding the complications of prolonged therapeutic exposure.

Spray LbL assembly was explored as a method for preventing the pH 6 destabilization of the underlying antibiotic film during assembly of the diclofenac/NSAID film due to the rapid kinetics and short time frame of the process. A representative chondroitin sulfate antibiotic-only film was used in these studies. The fast LbL spray process allows for the kinetic trapping of film components and does not allow significant film component interdiffusionfor the systems studied here. This composite architecture was found to have a film thickness of 3.00±0.16 μm, compared to 2.54±0.06 μm for a chondroitin sulfate spray antibiotic-only film (note that sprayed NSAID-only films have a thickness of 0.20±0.01 μm). Due to the short spray times of the NSAID architecture upon the antibiotic film, there was no significant decrease in vancomycin loading in composite films compared to antibiotic-only sprayed films. Spray LbL of antibiotic-only films has previously been shown to lead to short (4 hr) burst release of drug, which was also observed here. This composite architecture was found to address the need for immediate bacteria eradication in some cases and allow long term inflammation control, as seen in FIG. 6B. There was an NSAID release time of 13.9 days (twice as long as an NSAID-only film) with 36.2±4.4 μg/cm² diclofenac (5 times increased loading compared to an NSAID-only sprayed film) released in a nonlinear manner; 80% of the incorporated drug was released in the first 6 days. It is interesting that there was such a large increase in diclofenac loading during the spray process, which has previously been shown to lack the level of film component interdiffusion that is often visible in dip assembled films. However, the level of interpenetration of layers in the thin NSAID film may be enough to promote polyCD-vancomycin interactions, which are not as visible with thicker films. For all of the composite architectures explored in this Example, drug loading characteristics were consistent with the interaction studies completed prior to film assembly.

### Example 3

### Coating and Characterization of Layer-by-Layer Films on Medical Devices

In this Example, LBL architectures constructed according to Examples 2 and 3 were applied to several medical devices, including, but not limited to, IOLs, bandages, and sutures. Coating of these substrates demonstrates the versatility of these composite films in their ability to coat various medical device surfaces. The present invention, among other things, provides compositions and methods that can be applied to coatings for applications in personalized medicine, transdermal delivery, medical devices, nanoparticulate carriers, prosthetic implants, as well as small molecules for imaging, agriculture, and basic scientific research.

### Results

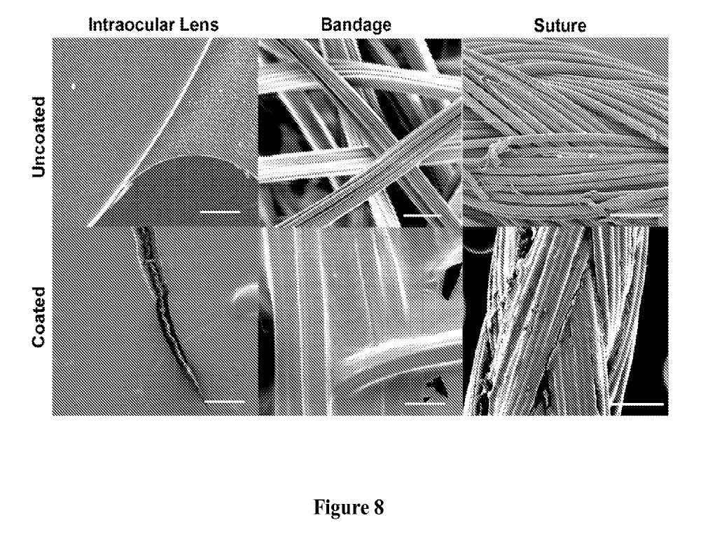

The therapeutic potential of the optimal dip and spray LbL architectures (whose release profiles are shown in FIG. 6) was assessed by applying these films to several medical device surfaces, including intraocular lenses (IOLs), bandages, and sutures. Scanning electron microscopy confirmed the successful coating of these devices, as seen in FIG. 8. The IOL was coated using dip LbL film assembly, while both the bandages and sutures were coated using spray assembly. In the uncoated IOL SEM image in FIG. 7, we see both the smooth lens region and the haptic. In the coated IOL image, a scratch was intentionally imaged to elucidate the existence of a smooth film on the IOL. Both the bandage and suture images clearly show the existence of film coating after the spray process on the substrates.

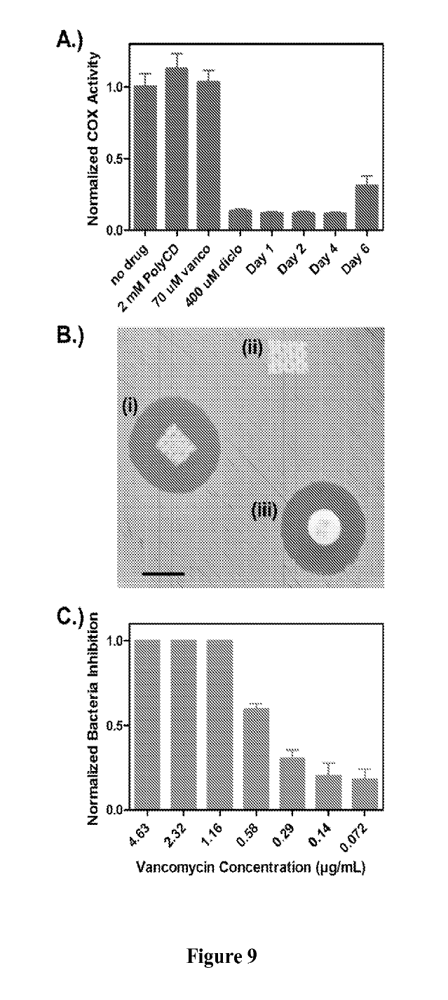

Therapeutic efficacy of bandages spray coated with the LbL film architecture whose release is shown in FIG. 6B was examined by challenging with specific infectious and inflammatory targets, namely S. aureus and COX. FIG. 9A shows the COX activity in response to diclofenac released from these coated bandages, along with several negative and positive controls. Film-released diclofenac was highly effective in inhibiting COX activity over the duration of its release. Vancomycin released from this coated bandage was also completely effective in inhibiting S. aureus growth in vitro, shown in FIG. 9B. The coated bandage has a surrounding zone of inhibition (ZOI) similar to a vancomycin control disk (30 μg); the ZOI is absent for the uncoated control bandage. Vancomycin released from a coated intraocular lens was also shown to completely maintain its native MIC against S. aureus (0.5-2 μg/mL) as shown in FIG. 9C; here the coating architecture was that of the dipped film release shown in FIG. 6A. Overall, the antibiotic and anti-inflammatory properties of the incorporated therapeutics were not affected by the composite film deposition and release process. These dual drug releasing films have great potential to be used in a variety of medical scenarios which would benefit from the localized delivery of both an antibiotic and an NSAID.

Furthermore, in compliance with FDA's standards, animal and/or clinical data will be obtained from an exemplary IOL system provided in the present invention. Nonclinical studies will be conducted. For example, a battery of in-vivo and in-vitro acute and chronic toxicity tests can be done in order to establish the biocompatibility of such an IOL system. Clinical studies on overall visual acuity, adverse reactions, postoperative complications, etc. will be conducted. Similar or improved data to the one of a commercial IOL is expected.

All literature and similar material cited in this application, including, patents, patent applications, articles, books, treatises, dissertations and web pages, regardless of the format of such literature and similar materials, are expressly incorporated by reference in their entirety. In the event that one or more of the incorporated literature and similar materials differs from or contradicts this application, including defined terms, term usage, described techniques, or the like, this application controls.

The section headings used herein are for organizational purposes only and are not to be construed as limiting the subject matter described in any way.

### OTHER EMBODIMENTS AND EQUIVALENTS

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

1. A decomposable film comprising:
a plurality of multi-layers of alternating first and second charges,
wherein the multi-layers comprise polyelectrolyte layers and one or more releasable agents; and
wherein decomposition of the film is characterized by sequential removal of at least a portion of the polyelectrolyte layers by alternating delamination of polyelectrolyte layers having the first charge and degradation of polyelectrolyte layers having the second charge, such that a controlled release of the at least one or more releasable agents is achieved.

2-12. (canceled)

13. The decomposable film of claim 1, wherein the one or more releasable agents are associated with a polyelectrolyte in the polyelectrolyte layers of the film.

14-17. (canceled)

18. The decomposable film of claim 1, wherein the one or more releasable agents comprise an anti-infective agent, an anti-flammatory agent or any combination thereof.

19. The decomposable film of claim 18, wherein the anti-infective agent is an antibiotic selected from the group consisting of a fluoroquinilone, macrolide, aminoglycoside, beta lactam, vancomycin and any combination thereof.

20. The decomposable film of claim 18, wherein the anti-inflammatory agent is selected from the group consisting of a corticosteroid, non-steroidal anti-inflammatory agent, mTOR inhibitor, calcineurin inhibitor, PI3K inhibitor, p38 inhibitor, JAK inhibitor, SYK inhibitor, HDAC inhibitor and any combination thereof.

21. The decomposable film of claim 1, wherein the film is deposited on a substrate.

22. The decomposable film of claim 21, wherein the substrate comprises at least a portion of a medical device.

23. The decomposable film of claim 21, wherein the substrate comprises at least a portion of an intraocular lens (IOL).

24. The decomposable film of claim 23, wherein the substrate comprises at least a portion of haptics of the IOL, at least a portion of an optic of the IOL or any combinations thereof.

25. The decomposable film of claim 23, wherein the substrate comprises a portion of an optic of the IOL.

26-28. (canceled)

29. An intraocular lens (IOL) system comprising:
an IOL and
one or more decomposable films deposited on the IOL,
wherein each decomposable film comprises a plurality of multi-layers of alternating first and second charges, and wherein the multi-layers comprise polyelectrolyte layers and one or more releasable agents;
wherein decomposition of the film is characterized by sequential removal of at least a portion of the polyelectrolyte layers by alternating delamination of polyelectrolyte layers having the first charge and degradation of polyelectrolyte layers having the second charge, such that a controlled release of the at least one or more releasable agents is achieved.

30. The IOL system of claim 29, wherein the one or more decomposable films are respectively deposited on at least a portion of haptics of the IOL, at least a portion of an optic of the IOL or any combinations thereof.

31. The IOL system of claim 29, wherein the IOL is foldable.

32. In a method of utilizing an IOL, which method comprising implanting the IOL, the improvement that comprises depositing one or more decomposable films on at least a portion of the IOL,
wherein each decomposable film comprises a plurality of multi-layers of alternating first and second charges, and wherein the multi-layers comprise polyelectrolyte layers and one or more releasable agents;
wherein decomposition of the film is characterized by sequential removal of at least a portion of the polyelectrolyte layers by alternating delamination of polyelectrolyte layers having the first charge and degradation of polyelectrolyte layers having the second charge, such that a controlled release of the at least one or more releasable agents is achieved.

33. The methods of claim 32, wherein the at least one or more releasable agents comprise an anti-inflammatory agent, such that inflammation after the IOL implantation is reduced as compared with that observed for an otherwise identical IOL lacking the decomposable film.

34. The methods of claim 32, wherein the at least one or more releasable agents comprise an antibiotic, such that infection after the IOL implantation is reduced as compared with that observed for an otherwise identical IOL lacking the decomposable film.

35. The methods of claim 32, further comprising no substantive alternation/modification of the IOL.

36. The methods of claim 32, further comprising no substantive alternation/modification of the IOL implantation.

37. A method of making a coated system comprising steps of:
associating one or more releasable agents within a decomposable film comprising a plurality of multi-layers of alternating first and second charges,
wherein the multi-layers comprise polyelectrolyte layers; and
wherein decomposition of the film is characterized by sequential removal of at least a portion of the polyelectrolyte layers by alternating delamination of polyelectrolyte layers having the first charge and degradation of polyelectrolyte layers having the second charge; and
depositing the film on a substrate.

38-44. (canceled)

45. A method of using a coated system comprising steps of:
providing a coated system comprising one or more decomposable films on a substrate
wherein each decomposable film comprises a plurality of multi-layers of alternating first and second charges, and wherein the multi-layers comprise polyelectrolyte layers and one or more releasable agents;
wherein decomposition of the film is characterized by sequential removal of at least a portion of the polyelectrolyte layers by alternating delamination of polyelectrolyte layers having the first charge and degradation of polyelectrolyte layers having the second charge; and
releasing the one or more releasable agents from the film.

46-51. (canceled)
